(19) United States
(12) Patent Application Publication
(10) Pub. No.: US 2024/0091034 A1
(43) Pub. Date: Mar. 21, 2024

(54) DEVICE AND METHOD FOR CLOSURE OF SINUS VENOSUS ATRIAL SEPTAL DEFECTS

(71) Applicant: INSTITUTE OF ADVANCED MEDICAL RESEARCH AND INNOVATIONS FORUM, Hyderabad (IN)

(72) Inventor: Joseph John Vettukattil, Grand Rapids, MI (US)

(21) Appl. No.: 17/768,030

(22) PCT Filed: Oct. 9, 2020
(86) PCT No.: PCT/IB2020/059512

§ 371 Date: Apr. 11, 2022

Related U.S. Application Data
(60) Provisional application No. 62/913,920, filed on Oct. 11, 2019.

(51) Int. Cl. A61F 2/86

(52) U.S. Cl. CPC A61F 2/86; A61F 2002/061

(57) ABSTRACT

The invention provides devices and methods for treating sinus venosus atrial septal defects. In an embodiment, the invention provides a sinus venosus atrial septal defect stent having a tubular body. The stent comprises a first end defining a proximal opening surrounded by a radially expandable skirt, a central conduit extending away from the skirt, and a second end defining a distal opening, distal from the first end and joined with the central conduit. The second end comprises a second diameter that is greater than the central diameter but less than the first diameter. The second end may be configured to anchor to the wall of the superior vena cava above the right atrium.

### CROSS-REFERENCE TO RELATED APPLICATIONS

This application is a National Stage Application under 35 U.S.C. § 371 of PCT Application No. PCT/IB2020/059512, filed Oct. 9, 2020, which claims priority from and the benefit of U.S. Provisional Patent Application No. 62/913,920 filed on Oct. 11, 2019, which are hereby incorporated by reference in their respective entireties.

### FIELD OF THE INVENTION

The present invention relates to the field of percutaneous interventions, and particularly to devices and methods for treating sinus venosus atrial septal defects.

### BACKGROUND

The mammalian heart includes left and right atria, and left and right ventricles. Oxygenated blood from the lungs, flowing through each of the four pulmonary veins, enters the left atrium, which pumps the blood to the left ventricle. The left ventricle pumps that oxygenated blood to the body. Deoxygenated blood returning from the body, flows from the superior vena cava and inferior vena cava and other veins, into the right atrium. The right atrium pumps the deoxygenated blood into the right ventricle, which subsequently pumps the deoxygenated blood to the lungs where it is oxygenated.

In a properly formed heart, the left atrium and the right atrium are separated by a wall known as the interatrial septum. Sometimes, the interatrial septum does not properly develop, or is malformed, and an atrial septal defect can result. This type of heart disorder can be described as an opening in the interatrial septum—providing fluid passage between the left atrium and the right atrium. The opening caused by the atrial septal defect causes comingling of oxygenated blood and deoxygenated blood, such that blood pumped from the left atrium to the body has reduced oxygen levels. These reduced oxygen levels can produce a variety of problems. Some atrial septal defects can lead to complications such as chest pain, arrhythmias, atrial fibrillation and sometimes heart failure.

FIGS. 1A and 1B comprise illustrations of a cross-section of a heart H having an atrial septal defect ASD. Heart H comprises left atrium LA, right atrium RA, left ventricle LV and right ventricle RV. Oxygenated blood from the lungs, flowing through each of the left pulmonary veins LV and the right pulmonary veins, which pumps oxygenated blood into the atrias through one or more pulmonary vein openings PV01, PV02 within the atrias.

The left ventricle LV pumps that oxygenated blood to the body. Deoxygenated blood returning from the body, flows from the superior vena cava SVC and inferior vena cava IVC and other veins, into the right atrium RA. The right atrium RA pumps the deoxygenated blood into the right ventricle RV, which subsequently pumps the deoxygenated blood to the lungs where it is oxygenated.

In a properly formed heart, all of the pulmonary vein openings PV01, PV02 would be located in the left atrium LA, so that oxygenated blood from the lungs flows directly into the left atrium LA.

As shown FIGS. 1A and 1B, the atrial septal defect ASD comprises a hole in the atrial septum—which allows passage of blood between one of the left and right atria LA, RA and the other of the left and right atria LA, RA.

Additionally, there are multiple types of atrial septal defects (ASDs). One type is a sinus venosus ASD (SVASD), which can form at the entry site of the superior vena cava SVC into the right atrium RA i.e. at or near the roof of right atrium RAR. This location also generally corresponds to where pulmonary veins join with the heart. Accordingly, as illustrated in more detail in FIGS. 2A and 2B, while one set of pulmonary veins (e.g. the left pulmonary veins LPV) have pulmonary vein openings LPV01, LPV02 properly formed within the left atrium LA, one or more of the other pulmonary veins (e.g. the right pulmonary veins RPV) have abnormally formed pulmonary vein openings RPV01, RPV02, RPV03 within the right atrium RA and in some situations, in proximity of the atrial septal defect ASD.

These abnormally formed pulmonary vein openings RPV01, RPV02, RPV03 cause oxygenated blood to enter the right atrium RA instead of the left atrium LA, which further intensifies the problem of less oxygenated blood being pumped from the left atrium LA to the body.

One solution for treating SVASD is via open heart surgery. However, such surgery is quite invasive, traumatic and can require significant recovery time. Accordingly, there is a need for alternative devices and methods for treating SVASD.

### SUMMARY

The invention provides devices and methods for treating sinus venosus atrial septal defects.

In an embodiment, the invention provides a sinus venosus atrial septal defect stent having a tubular body. The stent comprises (i) a first end defining a proximal opening surrounded by a radially expandable skirt comprising a self-expanding mesh, the skirt having a first diameter, the first end including a first exposed mesh rim disposed adjacent the skirt that is configured to anchor to the roof of the right atrium adjacent an entry site of a superior vena cava, (ii) a central conduit extending away from the skirt, the self-expanding mesh covered by a cover along the central conduit, the central conduit defining an opening in the cover distal from the skirt, the central conduit in fluid communication with the proximal opening, the central conduit having a central diameter that is less than the first diameter, the central conduit configured to be located in the superior vena cava, the central portion having a longitudinal axis, and (iii) a second end defining a distal opening, distal from the first end and joined with the central conduit, the second end including a second diameter that is greater than the central diameter but less than the first diameter, the second end including a second end portion covered by the cover, and a second exposed mesh rim that protrudes beyond the second end portion covered by the cover, the second exposed mesh rim configured to anchor to the wall of the superior vena cava above the right atrium.

In a particular embodiment of the stent, the first end, central conduit and second end are arranged to provide a first flow path for blood from the superior vena cava into the right atrium generally along the longitudinal axis and through the first end, central conduit and second end, and simultaneously provide a second flow path for blood around the central conduit, generally transverse to the longitudinal axis, from a pulmonary vein through an atrial septal defect and into a left atrium.

The stent may be configured such that the central conduit defines a fenestration through the cover so as to provide fluid communication between an interior of the central conduit and an exterior of the central conduit.

In an embodiment of the stent, the fenestration is between 1 mm and 10 mm in dimension.

In a specific embodiment of the stent, (i) the skirt includes a transition portion that transitions to the central conduit, (ii) the transition portion has a transition diameter less than the first diameter and greater than the central diameter, and (iii) a radiopaque marker is disposed on the transition portion to enable monitoring of the positioning of the stent in the right atrium.

The stent may be configured such that the central diameter is less than half the first diameter. In a more particular embodiment, the central diameter is less than half the second diameter.

The stent may be configured such that (i) the central conduit includes an interior lumen and an exterior surface, wherein the blood in the first flow path passes through the interior lumen, and (ii) the blood in the second flow path passes across the exterior surface.

In an exemplary embodiment of the stent, the first end, central conduit and second end form an hourglass shaped tubular body.

In another embodiment, the stent may be configured such, the second end includes a second end length, and the second exposed mesh rim is at least half the second end length and anchor the superior vena cava.

In a particular embodiment of the stent, the ratio of the first diameter to the second diameter to the central diameter is between 10:6:3 and 3:2:1.

The invention additionally provides a method of treating a sinus venosus atrial septal defect, the method comprising (i) advancing a self-expanding stent through a superior vena cava and at least partially into a right atrium, (ii) placing a covered central conduit of the stent between a pulmonary vein and an atrial septal defect, the covered central conduit including a longitudinal axis, (iii) locating an exposed mesh portion of the stent adjacent the superior vena cava so that the exposed mesh portion can anchor to the superior vena cava, (iv) pulling a skirt of the stent against the roof of the right atrium adjacent an entry site of the superior vena cava so that a mesh rim of the skirt can anchor to the right atrium; and (v) establishing a first flow path for blood from the superior vena cava into the right atrium generally along the longitudinal axis and through the covered central conduit, and simultaneously establishing a second flow path for blood around and exterior to the covered central conduit, generally transverse to the longitudinal axis, from a right pulmonary vein through an atrial septal defect and into the left atrium.

The method may additionally comprise applying tension to the stent such that that the mesh rim of the skirt expands radially outward to increase in diameter and engages the skirt against the roof of the right atrium.

In a method embodiment, (i) the central conduit includes a central diameter that is less than a diameter of the superior vena cava so that the blood of the second flow path can flow between the central conduit and a wall of the superior vena cava, and (ii) the mesh rim of the skirt expands radially to a first diameter that is at least twice the central diameter.

In another method embodiment (i) the exposed mesh portion is adjacent a second end covered portion, and (ii) the second end covered portion is of a second diameter that is larger than the central diameter, so that blood of the second flow path cannot flow between the second covered portion and the wall of the superior vena cava.

In a particular embodiment of the method, the second diameter is at least twice the central diameter.

In one method embodiment (i) the stent has an overall length from a first end adjacent the skirt to a second distal end adjacent the exposed mesh portion, and (ii) the central conduit has a conduit length that is at least half the overall length.

In a specific embodiment, the method may include comprising monitoring placement of a radiopaque marker disposed adjacent the skirt to position skirt against the roof of the right atrium adjacent an entry site of the superior vena cava.

### BRIEF DESCRIPTION OF THE ACCOMPANYING DRAWINGS

FIGS. 1A, 1B, 2A and 2B are illustrations of sinus venous atrial septal defects in a heart.

FIGS. 3 and 4 are illustrations of embodiments of stents in accordance with the teachings of the present invention, in expanded forms.

FIG. 5 is an illustration of any embodiments of a stent in accordance with the teachings of the present invention, in a collapsed form.

FIGS. 6A and 6B are partial section views of the stent in a collapsed form, being guided within the superior vena cava to the right atrium.

FIGS. 7A, 7B and 8 illustrate delivery and placement of the stent of the present invention within the heart.

FIGS. 9A, 9B and 9C illustrate placement and functionality of the stent within the heart, so as to enable closure of an SVASD, while simultaneously establish first and second flow paths for blood, through and around the stent, respectively.

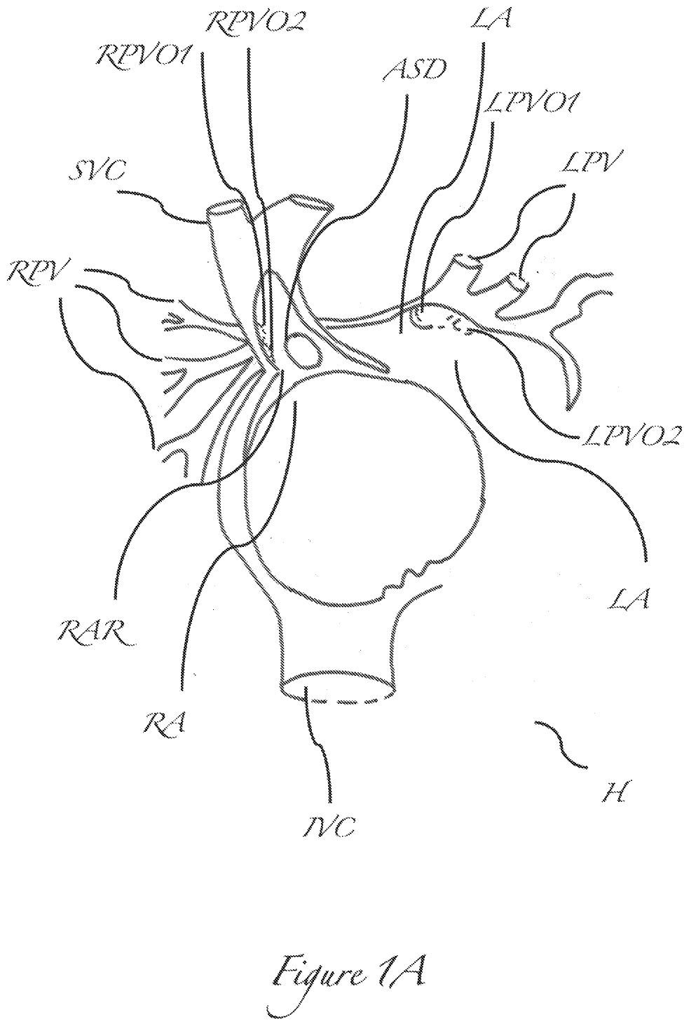

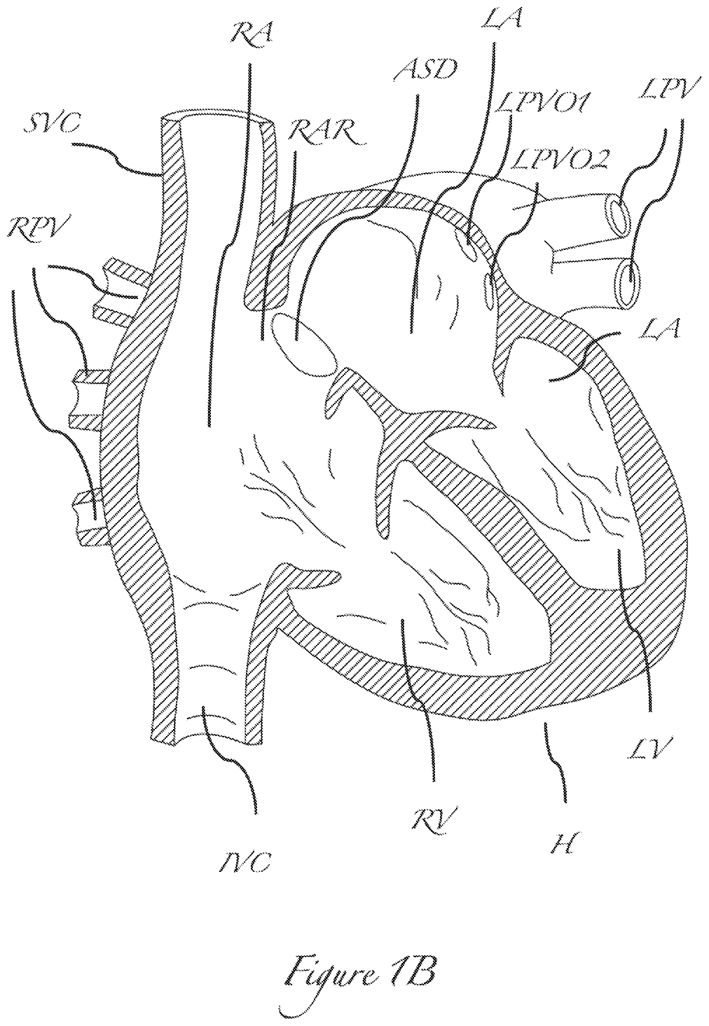

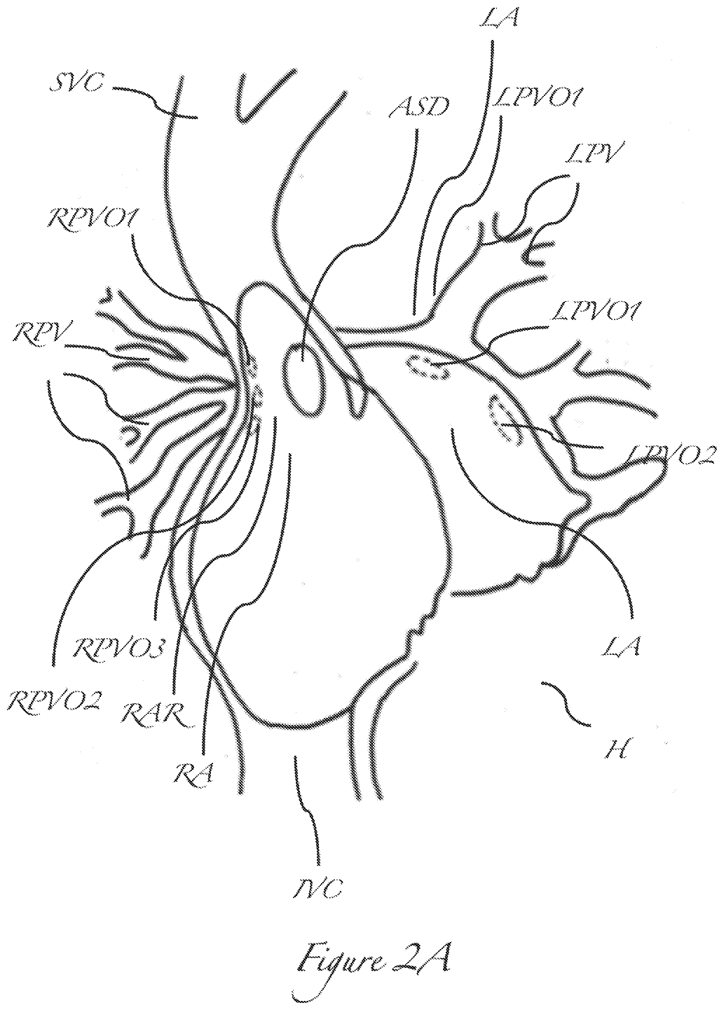

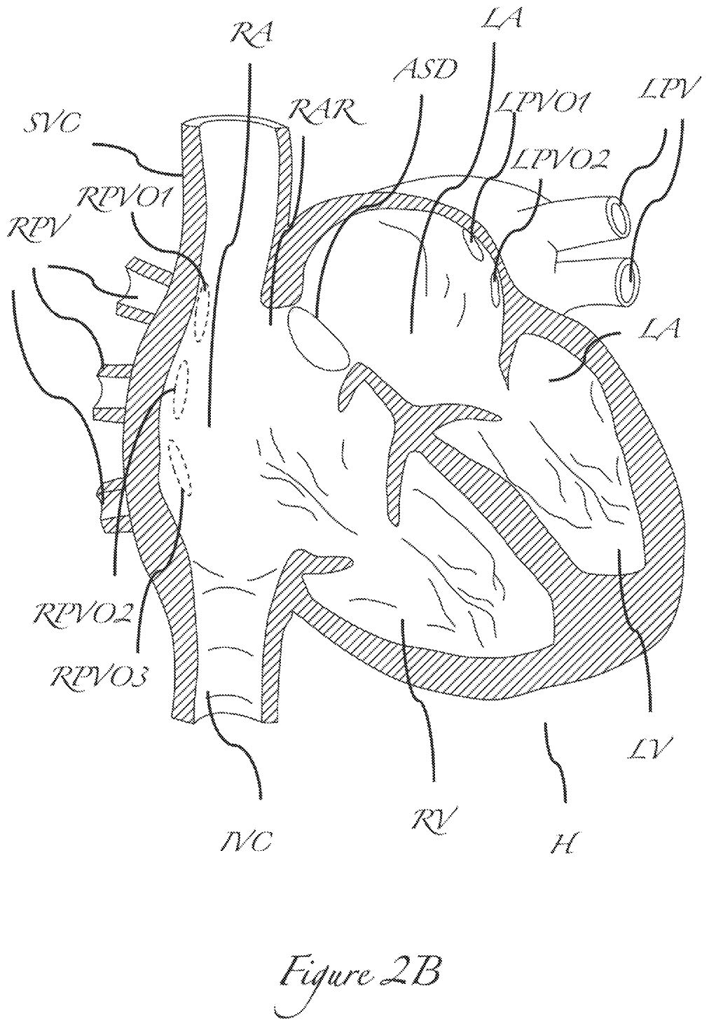

### DETAILED DESCRIPTION

The present invention provides devices and methods for treating sinus venosus atrial septal defects.

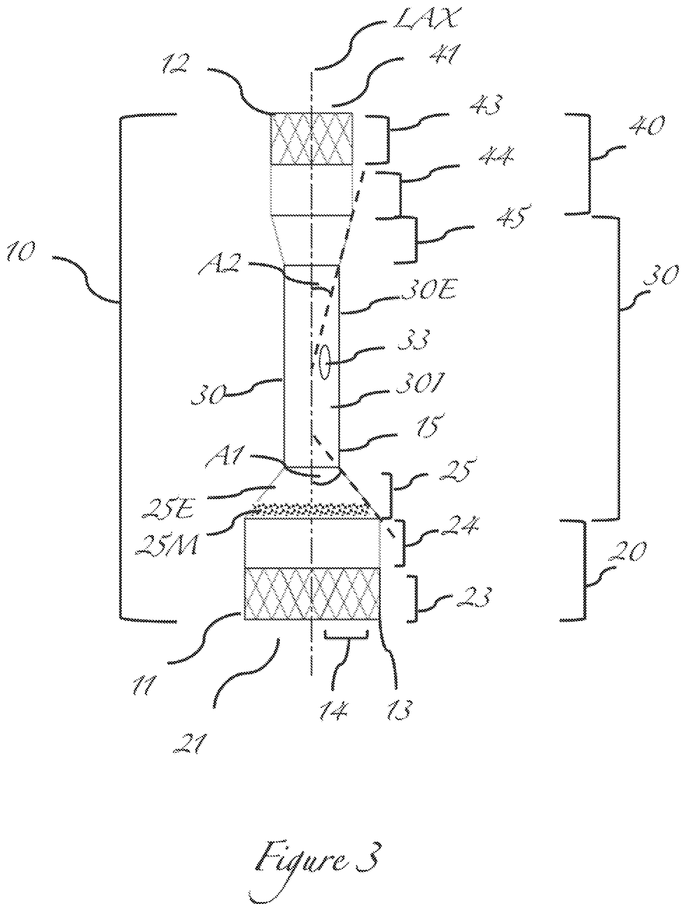

In an embodiment, the invention comprises a closure device 10 configured for closure of an atrial septal defect. FIGS. 3 to 9C illustrate embodiments of the closure device 10. In the illustrated embodiments, closure device 10 is a stent. The construction, configuration and functioning of closure device 10 is described in more detail below.

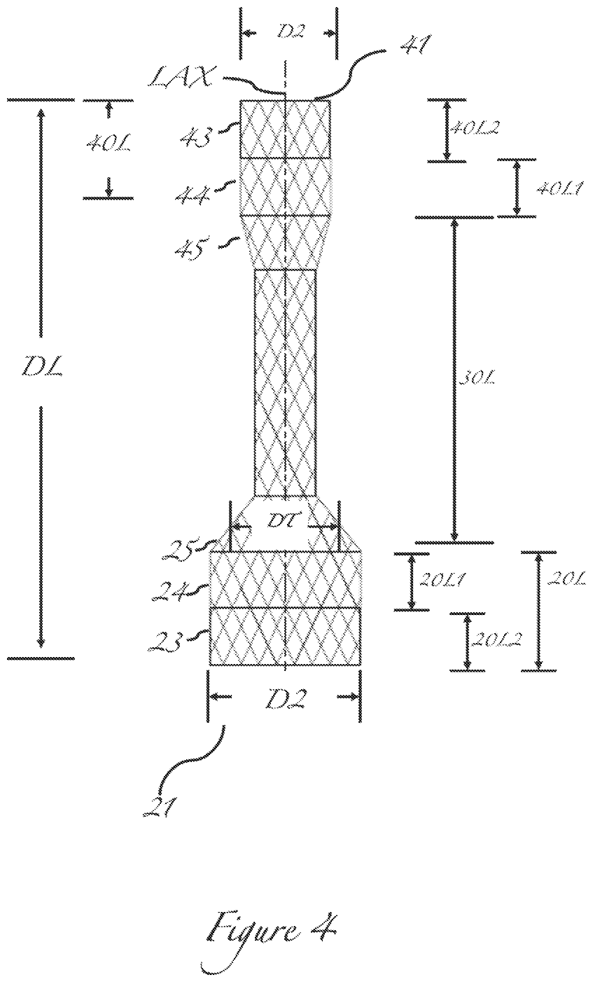

As shown in FIGS. 3 and 4, closure device 10 comprises a hollow, tubular stent 10. The stent 10 may be constructed from one or more flexible self-expanding shape memory materials. In an embodiment, stent 10 comprises an expandable mesh material 13 including multiple interlocking webs 14 or wires. The mesh 13 may be constructed from Nitinol, which is an alloy of nickel and titanium.

The mesh 13 can have a shape memory effect that returns the stent 10 to an original predetermined or preformed shape, after the stent is deformed—for example, when it is placed in a sheath or otherwise compressed or reduced in dimension. The mesh 13 may be used to form the perimeter of stent 10—thereby defining the overall shape and external configuration of said stent 10. In some embodiments, instead of the stent 10 being formed from a self-expanding mesh 13, a balloon can be inserted within stent 10 to expand the stent 10 to a predetermined shape, corresponding to the structures of the dimensions of the stent 10 disclosed herein.

The stent 10 can include a cover or sheath 15 that extends over multiple components and portions of the stent 10 along its length. This cover can be in the form of a film, sheet, coating, or other element that can cover the mesh 13 on the exterior, or optionally on the interior of the mesh 13. In some embodiments, the cover 15 can be constructed from polytetrafluoroethylene (PTFE), a polymeric film, a polymeric coating or other material. The cover 15 can be applied to the mesh 13 and to stent 10 when it is in an expanded condition. In use, for example, when the stent 10 is in a collapsed form, the cover 15 also can collapse, bunch up or reduce in dimension so that the stent 10 can be permitted to satisfactorily regain its expanded form when placed in a suitable location in the heart.

FIGS. 3 to 5 illustrate different configurations or forms of the stent 10. The stent 10 is shown in its expanded form in FIG. 3. Stent 10 can include a first end 11 and a second end 12. The first end 11 includes a flared or bell-shaped skirt 20. Skirt 20 may comprise a cylindrical, conical or frustoconical structure. The first end 11 can further define a proximal opening 21 that provides fluid communication into the stent 10 and with the remainder of the internal hollow tubular structure of the stent 10.

The skirt 20 may include a first exposed mesh rim 23 and a first covered portion 24 that are joined with or to the remainder of stent 10 through transition portion 25.

The mesh 13 can extend through and form portions of all of these different parts. The exposed mesh rim 23 can comprise the exposed mesh as shown. This mesh rim 23 can be configured to anchor to tissue in the heart. In connection with the current embodiment, this mesh rim 23 can specifically be configured to anchor to a roof or a portion of a roof of the right atrium RAR adjacent an entry site of a superior vena cava SVC of the heart H.

The skirt 20 at the first opening can have a first diameter D1. This diameter optionally can be at least 60 mm, at least 50 mm, at least 40 mm, at least 30 mm, at least 20 mm or at least 10 mm, between 10 mm and 50 mm, inclusive, or between 20 mm and 40 mm inclusive. This diameter can be optionally measured at the first opening 21, and can be the dimension of the first exposed mesh rim 23 at the opening 21.

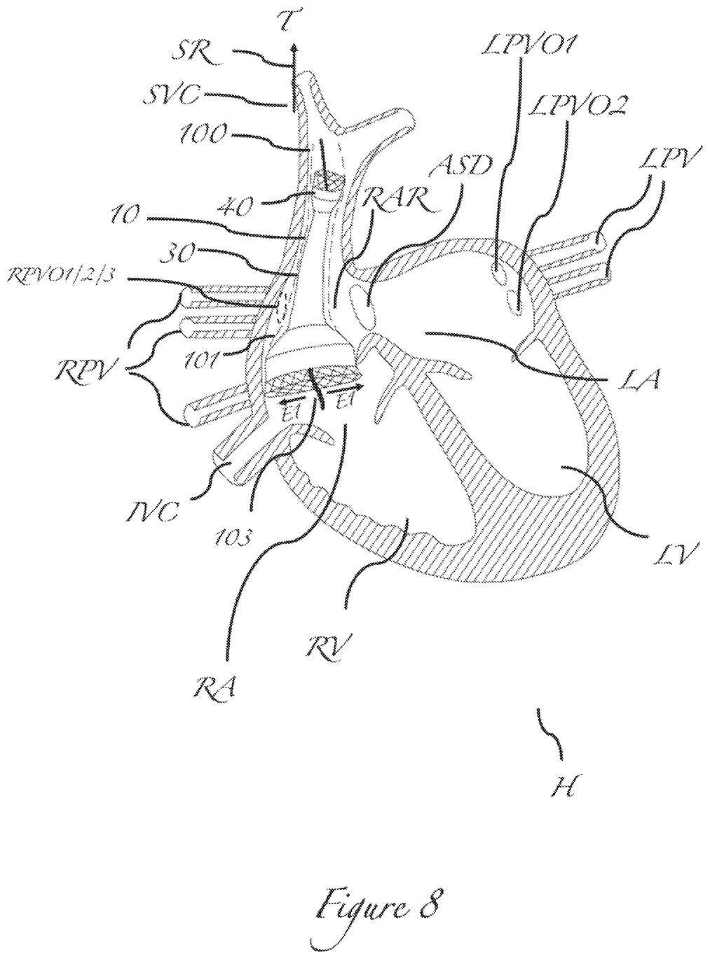

This diameter D1 can be modified or altered. For example, as described below, when a tension T, shown in FIG. 8, is exerted on the stent 10, and optionally removes a sheath 100 that is located about the stent 10, that first diameter D1 can be increased, with the skirt 20 expanding outward and increasing in dimension for example, at the opening 21. The skirt 20, and in particular the mesh portion 23 and the first covered portion 24, can radially expand outward, away from the longitudinal axis LAX of the stent 10 and of the central conduit 30. Upon such radial expansion, the mesh rim 23 can come into better contact and better engage the tissue in the roof RAR of the right atrium RA. This radial expansion also can provide a tighter seal around the stent 10 to prevent blood flow between the stent 10 and into the right atrium RA.

As shown in FIGS. 3 and 4, the first exposed mesh rim 23 can be disposed adjacent the first end covered portion 24. These portions can extend different lengths along the length 20L of the skirt 20. For example, the first exposed mesh rim 23 can extend along the length 20L2, while the first covered portion 24 can extend along a length 20L1. The length 20L1 can be about equal to the length 20L2. In other embodiments, these dimensions can differ. Optionally, the lengths 20L1 and 20L2 can be 5 mm to 25 mm, inclusive, 5 mm to 20 mm, inclusive, 10 mm to 15 mm, inclusive, or other lengths depending on the embodiment.

With reference to FIGS. 3 and 4, the first covered portion 24 of stent 10 can transition to the central conduit 30 at a transition portion 25 of the skirt 20. This transition portion 25 can taper in a reducing manner away from the proximal opening 21. The transition portion 25 optionally can include a decreasing transition diameter DT, which decreases in dimension away from the proximal opening 21. This transition diameter DT can be less than the first diameter D1, and greater than the center diameter DC of the central conduit 30. This transition portion 25 can be curved and/or angled outward and away from the longitudinal axis in transitioning from the central conduit 30 to the skirt 20. The precise amount of curving or angling can be dictated by the shape of the roof of the right atrium RAR of the patient in which the stent 10 is to be implanted.

Optionally, the transition portion 25 and/or another portion of the skirt 20 can include a radiopaque marker 25M. This radiopaque marker 25M can assist a surgeon in viewing and monitoring placement of the skirt 20 in a precise location relative to the right atrium RA, and in particular relative to the roof of the right atrium RA to plug the entry site of the superior vena cava SVC into the right atrium RA as described below. In other embodiments, other types of markers can be used instead of a radiopaque marker.

Returning to FIG. 3, the exterior surface 25E of the transition portion 25, also referred to as a first transition portion, can be covered with cover 15. The first transition portion 25 can be angled at an angle A1 relative to the longitudinal axis LAX of the stent 10 and of the central conduit 30. This angle A1 optionally can be at least 10°, 25°, 30°, 45°, 60°, between 10° and 55°, inclusive, between 10° and 45°, inclusive, between 30° and 50°, inclusive, between 40° and 50° inclusive, or about 45°. This angle can be selected depending on a particular angle of the roof RAR of the right atrium RA of a particular patient. The stent 10 can be custom manufactured to select this angle or tapered portion.

The transition portion 25 transitions to the central conduit 30. The central conduit 30 can be in the form of a substantially cylindrical element, extending from the first transition portion 25 to a second transition portion 45, away from the skirt 20 and toward the plug portion 40 (also referred to as the second end). The self-expanding mesh 13 can be disposed in the interior of the central conduit 30, with the external wall or external peripheral surface being covered by cover 15. The central conduit 30 can include an outer wall 34 that is generally of a cylindrical shape. The outer wall 34 can be substantially parallel to the longitudinal axis LAX of the stent 10 and the conduit 30. In an exemplary embodiment, the outer wall 34 of the central conduit 30 can be substantially parallel to the longitudinal axis LAX along a majority of the length 30L of the central conduit 30. The wall 34 also can be substantially parallel to, but radially disposed outward from, the longitudinal axis LAX for a length that is at least ⅓, ½, ⅔s, ¼, between ¼ and ¾, between ⅓ and ⅔s the overall length OL of the stent 10, as shown in FIG. 4.

With reference to FIG. 3, the central conduit 30 optionally can define an opening 33 in the cover 15, that is distal from the skirt 20 and distal from the plug portion 40. This opening, also referred to as a fenestration, can provide fluid communication between the interior 301 of the central conduit 30 and an exterior 30E of the central conduit 30. This opening can thereby allow a small amount of blood to be transferred between the interior and exterior of the central conduit in patients with an increased pressure within the right atrium RA (i.e. functions as a pressure release when pressure within the right atrium RA exceeds a predefined pressure, or exceeds a pressure in the left atrium LA). This opening 33 optionally can be between and 10 mm, inclusive, between 2 mm and 8 mm, inclusive, or about 2 mm in dimension.

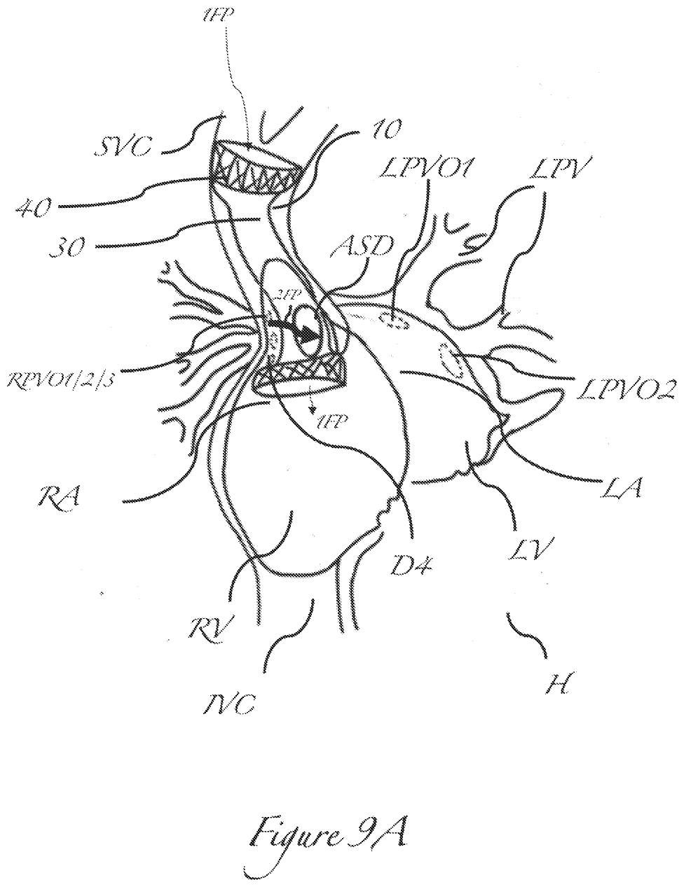

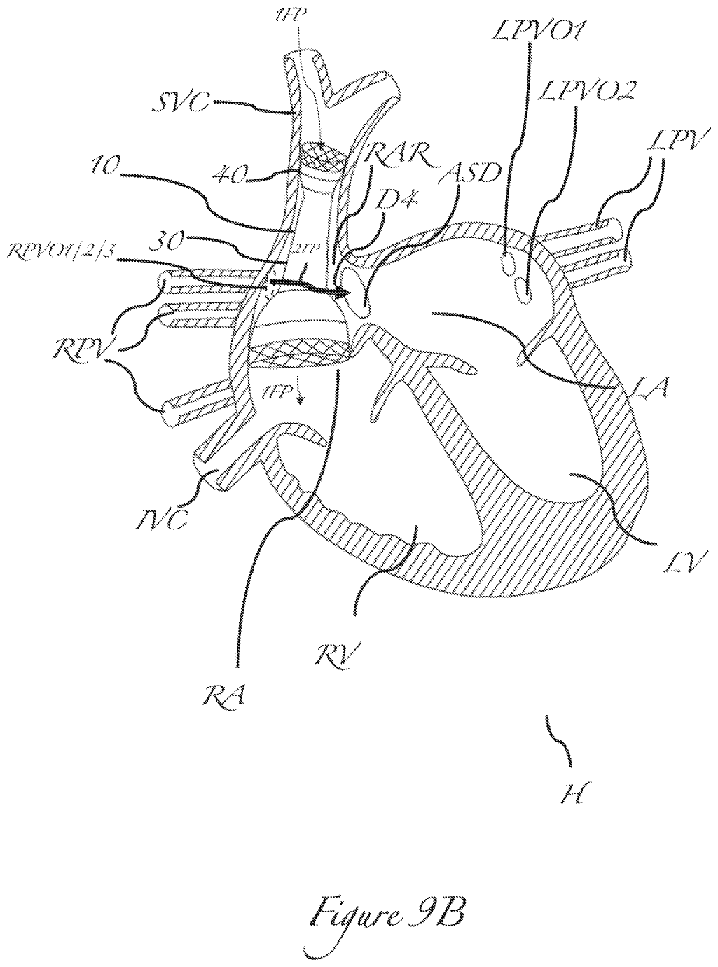

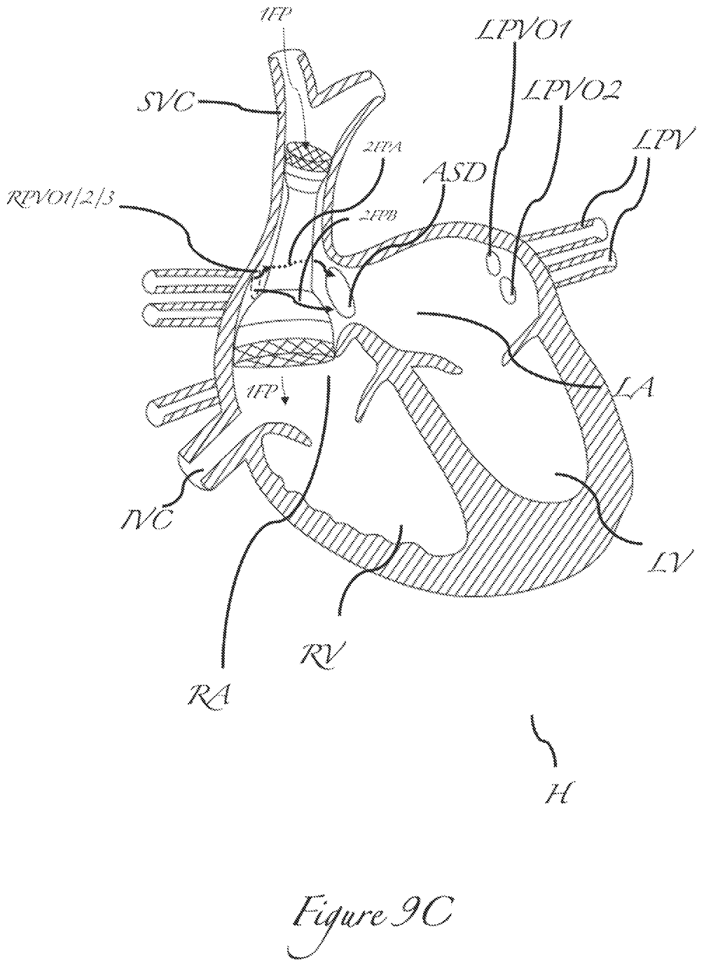

The central conduit 30 may be in fluid communication with the proximal opening 21 as well as the distal opening 41 of the stent 10. In an embodiment, central conduit 30 provides a fluid conduit or passage between the proximal opening 21 and distal opening 41 of stent 10. The central conduit 30, as mentioned above may have a central diameter DC. The central diameter optionally may be 5 mm to 20 mm, inclusive, 5 mm to 15 mm, inclusive, 10 mm to 20 mm, inclusive or other dimensions. Generally, this central diameter DC is a diameter that is less than the diameter of the superior vena cava SVC within which it is placed. In this manner, portions of a second flow path 2FP (described in more detail below in connection with FIGS. 9A to 9C) can flow between an external wall of the central conduit DC and the interior wall of the superior vena cava SVC. As an example (and as shown in FIG. 9C), the second flow path 2FP of blood can flow in a sub-flow path 2FPA and a separate sub-flow path 2FPB, around the central conduit when flowing from the openings of right pulmonary veins RPV through the atrial septal defect ASD and into the left atrium LA.

The central conduit 30 can have different dimensional relations relative to other parts of the stent 10. For example, the central conduit 30 can have a central diameter DC that is less than the first diameter D1 of the proximal opening 21 and also is less than the second diameter D2 of the distal opening 41. The central diameter DC optionally can be less than half the first diameter D1. The central diameter DC also can be less than half the second diameter D2. In other embodiments, other dimensional relationships between the central diameter DC and the other diameters can be selected depending on the specific embodiment or functionality.

In some cases, to facilitate blood flow through the superior vena cava SVC, in particular through the stent 10 into the right atrium RA, yet still allow blood flow around the central conduit 30 from a right pulmonary vein RPV through the atrial septal defect ASD and into the right atrium RA, the diameters of the different stent parts can be in particular ratios. As an example, the ratio of the first diameter D1 to the second diameter D2 to the central diameter DC can be between 10:6:3 and 3:2:1, or between 2:2:1 and 5:3:2, or other ratios depending on the construction and the heart structure.

The central conduit 30 can be joined with the second transition portion 45. This portion 45 can extend outward, increasing in diameter or dimension, away from the central conduit 30 toward the distal opening 41. This second transition portion 45 can include an exterior surface that is angled outwardly from the longitudinal axis LAX at an angle A2. This angle A2 can be less than the angle A1, optionally between 5° and 20°, inclusive, between 10° and 30°, inclusive, or between 5° and 20°, inclusive.

With reference to FIGS. 3 and 4, the second transition portion 45 may be joined with the second end portion 44 adjacent the second end. The second end 12 also can include a second exposed mesh rim 43 that protrudes or extends beyond the second end portion 44 that is covered by the cover 15.

The second end portion 44 and second exposed mesh rim 43 may form part of the plug portion 40 that fits within the superior vena cava SVC. The second end, or plug portion 40, can include a second length 40L. The second end portion 44 can include a length 40L1. The second exposed mesh rim can be of a length 40L2 that is at least half the second length or plug length 40L, to anchor to the superior vena cava SVC. The length 40L1 can be about equal to the length 40L2. Optionally, the lengths 40L1 and 40L2 can be 5 mm to 25 mm, inclusive, 5 mm to 20 mm, inclusive, 10 mm to 15 mm, inclusive, or other lengths depending on the application.

The plug portion 40 or the second end in general can define the distal opening 41 as described above. The second exposed mesh rim 43 can be configured to anchor to the wall of superior vena cava SVC, above the right atrium RA and above the right atrium roof RAR. By anchoring to a wall, tissue or other structure, it is contemplated that an element may be sutured or otherwise joined with the wall of the superior vena cava SVC, or optionally scar tissue forms over or around the mesh to embed the exposed mesh rim and thereby secure the mesh to the wall of the SVC.

Optionally, as shown in FIGS. 3 and 4, the stent 10 with its skirt 20, central conduit 30 and plug portion 40, can form an hourglass shaped tubular body. This hourglass shape can be helpful to effectively engage the interior wall of the superior vena cava SVC at the plug portion 40 or the second end 12, and simultaneously engage the roof of the right atrium RAR to provide a conduit through the interior of the stent 10 for a first flow path of blood to flow through, communicating from the superior vena cava SVC above the stent 10 directly into the right atrium RA below the stent 10. Further, this hourglass shape, with its reduced diameter central conduit, also establishes and allows another second flow path for blood around an exterior surface of the central conduit 30, without passing through the stent 10, and generally transverse to the longitudinal axis LAX of stent 10.

The second flow path 2FP can provide communication between an opening of right pulmonary vein RPV (for example an opening into right atrium RA within or in the vicinity of the atrial septal defect ASD), around the exterior of the central conduit 30, through the atrial septal defect ASD and into the left atrium LA. In this manner, the atrial septal defect ASD is not closed and remains open and unplugged. However, fluid communication between the superior vena cava SVC and the atrial septal defect ASD is interrupted and generally terminated.

Generally, the first end 11, central conduit 30 and second end 12 of the stent 10 are arranged to establish and provide a first flow path 1FP for blood from the superior vena cava SVC into the right atrium RA along the longitudinal axis LAX and passing through the first end 11, central conduit 30 and second end 12 within stent 10. These components of the stent 10 also simultaneously establish and provide a second flow path 2FP for blood around and external to the central conduit 30, in a direction that is generally transverse to the longitudinal axis LAX, from openings corresponding to one or more right pulmonary veins RPV through the atrial septal defect ASD and into the left atrium LA.

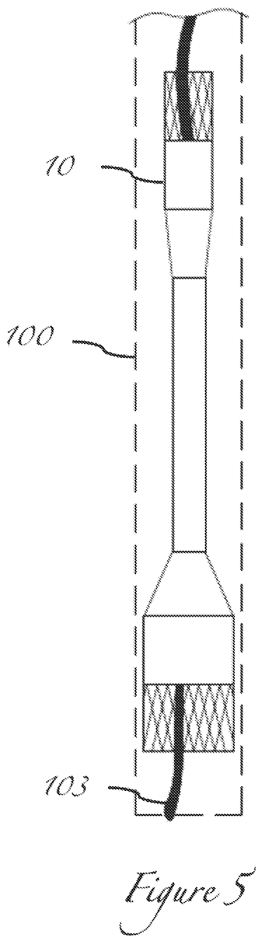

Reference to FIGS. 5 to 9C may be made to describe a method of implanting the stent 10 to treat a sinus venosus atrial septal defect. For the purposes of describing the methods of the present invention, it would be understood that stent 10 may have any one or more of the configurations described in detail above. For example, the stent 10 can be constructed from a self-expanding mesh. The mesh, its cover and various structures can be collapsed to a collapsed form as shown in FIG. 5. In its collapsed form, the stent 10 can be placed within in a sheath 100. The sheath 100 can be sized as shown, or of different dimensions sufficient to radially compress stent 10 in an inward direction to enable introduction into and travel through the superior vena cava SVC of the heart H. A guide wire 103 can be associated with the stent 10 and the sheath 100—and may be used to guide stent 10 and sheath 100 through the superior vena cava SVC. In an embodiment, guide wire 103 passes through first end 11, central conduit 30 and second end 12 of stent 10 and is used to guide stent 10 to an appropriate position within the heart H.

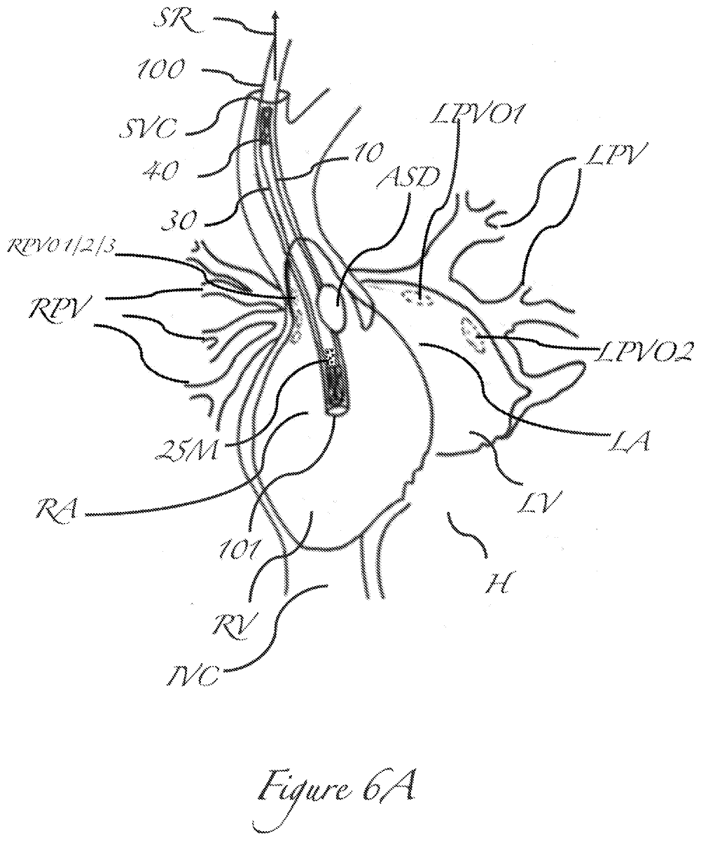

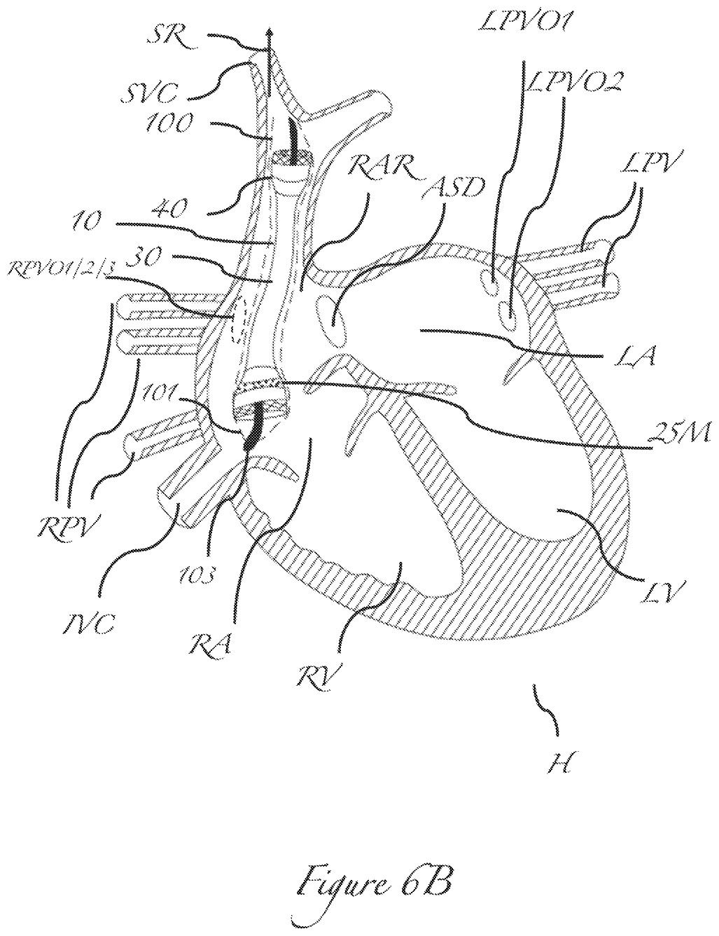

As shown in FIG. 6B, a guide wire 103 can be extended through the superior vena cava SVC, past the right pulmonary veins RPV and past the atrial septal defect ASD, into the right atrium RA, optionally without ever passing through the left atrium LA or the atrial septal ASD. With the guide wire 103 so placed, the sheath 100 with the stent 10 in a collapsed form therein can be advanced along the guide wire 103 until located in the position shown approximately in FIG. 6A and/or FIG. 6B. As shown in FIGS. 6A and 6B, the plug portion 40 of stent 30 is positioned within the superior vena cava SVC, such that the central conduit 30 is generally adjacent to the right pulmonary veins RPV and the atrial septal defect ASD, while the skirt 20 in its collapsed form is placed at least partially in the right atrium RA adjacent the right atrium roof RAR. Where included, the marker 25M can be used by a surgeon to determine the precise placement of the skirt 20 relative to the atrium and entry site of the superior vena cava SVC into the right atrium RA.

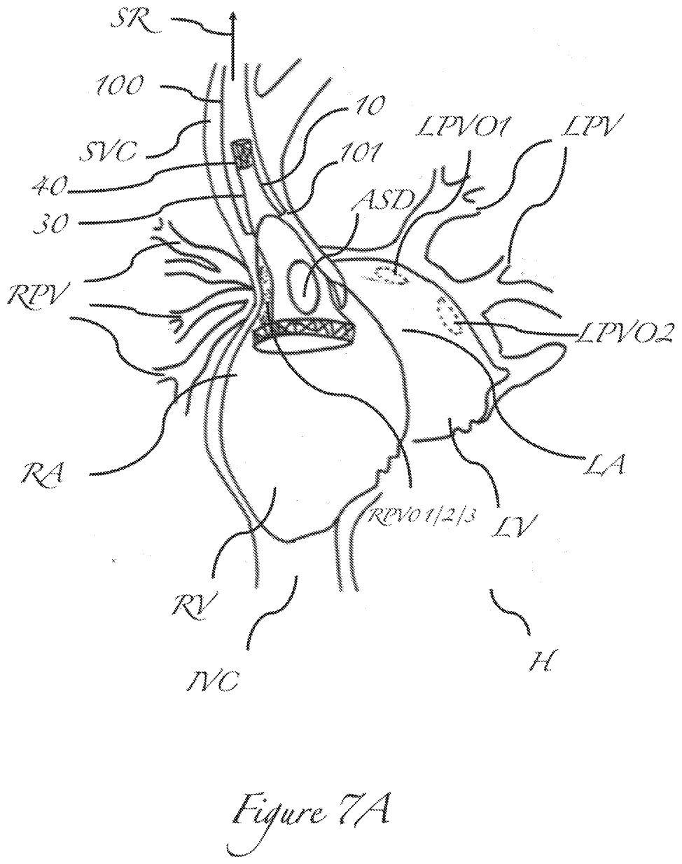

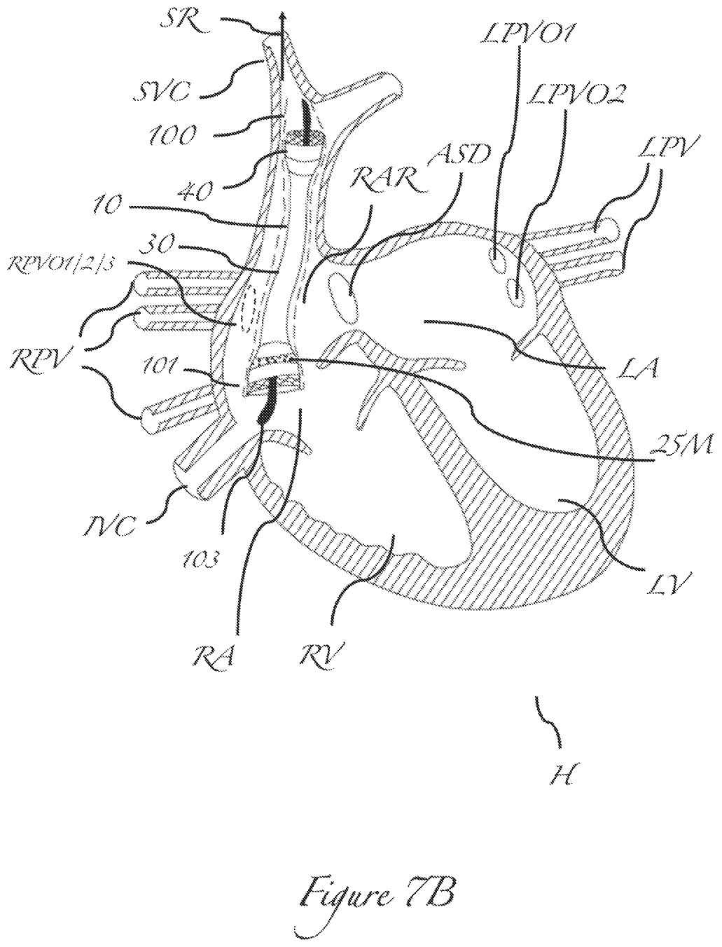

As shown in FIGS. 7A and 7B, the stent 10 can be further placed with its various structures and more precisely aligned in the heart H. In particular, the sheath 100 can be slightly withdrawn from the stent 10 in direction SR. As this sheath is removed or withdrawn in the direction SR, the end 101 of the sheath 100 uncovers the skirt 20. As withdrawal of sheath 100 uncovers the stent 10, the self-expanding mesh in the skirt 20 expands such that the dimension of the skirt increases from a reduced dimension to an expanded dimension. For example, the first end 21 can expand to the first diameter D1, from a smaller compressed diameter. The other components adjacent the skirt 20 likewise can expand to their respective uncompressed diameters. The transition portion 25 also can expand as illustrated.

Additionally, as shown in FIGS. 9A and 9B, the stent 10 may be positioned such that there is an offset gap D4 between of the skirt 20 and the roof of the right atrium RAR. In particular the skirt can be spaced by that gap or a corresponding distance D4 from a portion of the roof RAR at the entry site of the superior vena cava SVC into the right atrium RA. In some embodiments, this placement of the stent 10 can be corrected or adjusted so that there is no gap D4 between the skirt and the entry site of the superior vena cava into the right atrium. The reason this gap D4 may be eliminated or reduced is so that blood does not enter the right atrium from the right pulmonary veins or atrial septal defect ASD through that gap D4.

To adjust the placement of the skirt 20 and the stent 10 in general, to thereby close gap D4, a user can monitor the marker 25M, and move the skirt 20 upward by pulling the stent and sheath upward in direction SR so that the skirt is brought against and engages the right atrium roof RAR, as shown in FIG. 8 and in FIGS. 9A to 9C. To further close that gap, a user can exert tension T on the stent, via the guide wire 103 and/or the sheath 100.

When tension T is applied, the skirt 20, for example the first exposed mesh rim 23, expands outward in direction E1, radially away from the longitudinal axis LAX of the stent 10. As this occurs, the mesh rim 23 can more fully engage the roof so that exposed mesh portion can anchor to the right atrium, and optionally to the roof of the right atrium RAR or at the entry site of the superior vena cava SVC to the right atrium RA.

With the tension T applied, the stent and skirt can further lodge in or engage the entry site of the superior vena cava to the right atrium, thereby effectively occluding that entry site. The transition portion 25 and/or the first and portion 24, covered with the cover, can engage the tissue at that site thereby sealing it. Likewise, the mesh rim can extend outward and radially around that longitudinal axis in that area of the site.

With the skirt 20 properly placed, and optionally expanded, the user can further withdraw the sheath 100 and remove it from the stent 10. As the sheath 100 is removed, the remainder of the stent transitions from the collapsed form to the expanded form as shown in FIGS. 9A to 9C. The second exposed mesh rim 43, also referred to as an exposed mesh portion, is lodged in the superior vena cava SVC as shown. Due to the greater diameter of the plug portion 40, the second end covered portion 44 expands outward and engages the wall of the superior vena cava SVC.

With this engagement, the plug portion 40 can effectively engage the wall of the superior vena cava to prevent blood flow from passing by the plug portion. The mesh rim also is configured to anchor to the superior vena cava, via sutures or other attachments, or simply by growth of tissue over the exposed mesh rim. The skirt 20 remains disposed in the entry site of the superior vena cava SVC into the right atrium RA and occludes that site.

With further reference to FIGS. 9A to 9C, the placement of the stent 10 establishes a first flow path for blood 1FP from the superior vena cava SVC into the right atrium RA, generally along and/or parallel to the longitudinal axis LAX. The blood thus can flow along this path 1FP through the interior of the stent, for example through the interior of the plug portion 40, through the interior of the central conduit 30 and through the interior of the skirt 20, and into the right atrium RA. Due to the occlusion of the superior vena cava SVC, external to the stent, above the right pulmonary veins RPV and below the right pulmonary veins, at the right atrium roof RAR, the stent also simultaneously establishes a second flow path 2FP for blood to flow from one or more right pulmonary veins RPV through an atrial septal defect ASD and into the left atrium LA.

As blood flows along the second flow path 2FP, the blood can be bifurcated into first and second sub paths 2FPA and 2FPB. These paths can go on opposite sides S1 and S2 of the longitudinal axis LAX, generally around the central conduit 30. Again because the central conduit is thinned or of a reduced diameter from the first and second ends of the stent, this allows the blood to flow around that part of the stent. In other words, the central conduit allows fluid communication between the right pulmonary veins RPV and the atrial septal defect ASD, while preventing that blood from entering the right atrium RA via the skirt 20. Generally, the second flow paths 2FPA and 2FPB may be transverse to the longitudinal axis LAX of the stent 10.

In some cases, the flow can be directed along various paths that are at various angles relative to that longitudinal axis LAX. Generally however, the flow is not substantially parallel to the longitudinal axis LAX. Further, the larger diameter or dimensioned plug portion 40 and skirt portion 20 of the stent 10 effectively prevent blood flowing along the second flow path 2FP from flowing between the stent and the wall of the superior vena cava and/or the right atrium roof. In turn, the second flow path 2FP of blood is isolated and redirected again from the pulmonary veins through the atrial septal defect ASD, which remains open, and into the left atrium LA.

For the purposes of the present description, directional terms, such as “vertical,” “horizontal,” “top,” “bottom,” “upper,” “lower,” “inner,” “inwardly,” “outer” and “outwardly,” are used to assist in describing the invention based on the orientation of the embodiments shown in the illustrations. The use of directional terms should not be interpreted to limit the invention to any specific orientation(s).

In addition, when a component, part or layer is referred to as being “joined with,” “on,” “engaged with,” “adhered to,” “secured to,” or “coupled to” another component, part or layer, it may be directly joined with, on, engaged with, adhered to, secured to, or coupled to the other component, part or layer, or any number of intervening components, parts or layers may be present. In contrast, when an element is referred to as being “directly joined with,” “directly on,” “directly engaged with,” “directly adhered to,” “directly secured to,” or “directly coupled to” another element or layer, there may be no intervening elements or layers present. Other words used to describe the relationship between components, layers and parts should be interpreted in a like manner, such as “adjacent” versus “directly adjacent” and similar words. As used herein, the term “and/or” includes any and all combinations of one or more of the associated listed items.

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments.

For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative.

Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims.

Any reference to claim elements in the singular, for example, using the articles “a,” “an,” “the” or “said,” is not to be construed as limiting the element to the singular. Any reference to claim elements as “at least one of X, Y and Z” is meant to include any one of X, Y or Z individually, and any combination of X, Y and Z, for example, X, Y, Z; X, Y; X, Z; and Y, Z.

Additionally, the invention illustratively disclose herein suitably may be practiced in the absence of any element which is not specifically disclosed herein—and in a particular embodiment that is specifically contemplated, the invention is intended to be practiced in the absence of any one or more element which are not specifically disclosed herein.

We claim:

1. A sinus venosus atrial septal defect stent having a tubular body, and comprising:
a first end defining a proximal opening surrounded by a radially expandable skirt comprising a self-expanding mesh, the skirt having a first diameter, the first end including a first exposed mesh rim disposed adjacent the skirt that is configured to anchor to the roof of the right atrium adjacent an entry site of a superior vena cava;
a central conduit extending away from the skirt, the self-expanding mesh covered by a cover along the central conduit, the central conduit defining an opening in the cover distal from the skirt, the central conduit in fluid communication with the proximal opening, the central conduit having a central diameter that is less than the first diameter, the central conduit configured to be located in the superior vena cava, the central portion having a longitudinal axis; and
a second end defining a distal opening, distal from the first end and joined with the central conduit, the second end including a second diameter that is greater than the central diameter but less than the first diameter, the second end including a second end portion covered by the cover, and a second exposed mesh rim that protrudes beyond the second end portion covered by the cover, the second exposed mesh rim configured to anchor to the wall of the superior vena cava above the right atrium;
wherein the first end, central conduit and second end are arranged to provide a first flow path for blood from the superior vena cava into the right atrium generally along the longitudinal axis and through the first end, central conduit and second end, and simultaneously provide a second flow path for blood around the central conduit, generally transverse to the longitudinal axis, from a pulmonary vein through an atrial septal defect and into a left atrium.

2. The stent as claimed in claim 1, wherein the central conduit defines a fenestration through the cover so as to provide fluid communication between an interior of the central conduit and an exterior of the central conduit.

3. The stent as claimed in claim 2, wherein the fenestration is between 1 mm and 10 mm in dimension.

4. The stent as claimed in claim 1, wherein:
the skirt includes a transition portion that transitions to the central conduit;
the transition portion has a transition diameter less than the first diameter and greater than the central diameter; and
a radiopaque marker is disposed on the transition portion to enable monitoring of the positioning of the stent in the right atrium.

5. The stent as claimed in claim 1, wherein the central diameter is less than half the first diameter.

6. The stent as claimed in claim 5, wherein the central diameter is less than half the second diameter.

7. The stent as claimed in claim 1, wherein:
the central conduit includes an interior lumen and an exterior surface, wherein the blood in the first flow path passes through the interior lumen; and
the blood in the second flow path passes across the exterior surface.

8. The stent as claimed in claim 7, wherein the first end, central conduit and second end form an hourglass shaped tubular body.

9. The stent as claimed in claim 1, wherein:
the second end includes a second end length; and
the second exposed mesh rim is at least half the second end length and anchor the superior vena cava.

10. The stent as claimed in claim 9, wherein the ratio of the first diameter to the second diameter to the central diameter is between 10:6:3 and 3:2:1.

11. A method of treating a sinus venosus atrial septal defect, the method comprising:
advancing a self-expanding stent through a superior vena cava and at least partially into a right atrium;
placing a covered central conduit of the stent between a pulmonary vein and an atrial septal defect, the covered central conduit including a longitudinal axis;
locating an exposed mesh portion of the stent adjacent the superior vena cava so that the exposed mesh portion can anchor to the superior vena cava; and
pulling a skirt of the stent against the roof of the right atrium adjacent an entry site of the superior vena cava so that a mesh rim of the skirt can anchor to the right atrium;
establishing a first flow path for blood from the superior vena cava into the right atrium generally along the longitudinal axis and through the covered central conduit, and simultaneously establishing a second flow path for blood around and exterior to the covered central conduit, generally transverse to the longitudinal axis, from a right pulmonary vein through an atrial septal defect and into the left atrium.

12. The method as claimed in claim 11, comprising:
applying tension to the stent such that that the mesh rim of the skirt expands radially outward to increase in diameter and engages the skirt against the roof of the right atrium.

13. The method as claimed in claim 12, wherein:
the central conduit includes a central diameter that is less than a diameter of the superior vena cava so that the blood of the second flow path can flow between the central conduit and a wall of the superior vena cava; and
wherein the mesh rim of the skirt expands radially to a first diameter that is at least twice the central diameter.

14. The method as claimed in claim 13, wherein:
the exposed mesh portion is adjacent a second end covered portion; and
the second end covered portion is of a second diameter that is larger than the central diameter, so that blood of the second flow path cannot flow between the second covered portion and the wall of the superior vena cava.

15. The method as claimed in claim 14, wherein the second diameter is at least twice the central diameter.

16. The method as claimed in claim 11, wherein:
the stent has an overall length from a first end adjacent the skirt to a second distal end adjacent the exposed mesh portion; and
the central conduit has a conduit length that is at least half the overall length.

17. The method as claimed in claim 11, comprising monitoring placement of a radiopaque marker disposed adjacent the skirt to position skirt against the roof of the right atrium adjacent an entry site of the superior vena cava.
